(19) United States
(12) Patent Application Publication
(10) Pub. No.: US 2026/0345527 A1
(43) Pub. Date: Nov. 5, 2026

(54) COMPOSITION CONTAINING COLCHICINE AND METFORMIN FOR PREVENTING AND TREATING NEURODEGENERATIVE DISEASES

(71) Applicants: Ju Ho YOUN, Seoul (KR); Sun Ah HAM, Changwon-si (KR)

(72) Inventors: Ju Ho YOUN, Seoul (KR); Sun Ah HAM, Changwon-si (KR)

(21) Appl. No.: 19/125,608

(22) PCT Filed: Oct. 29, 2023
(86) PCT No.: PCT/KR2023/016948

§ 371 Date: Apr. 29, 2025

(30) Foreign Application Priority Data
Nov. 1, 2022 (KR) 10-2022-0144058

(51) Int. Cl. A61K 31/165; A61K 31/122; A61K 31/155; A61K 31/198; A61K 31/205; A61K 31/355; A61K 31/4045; A61K 31/593; A61K 33/06; A61P 25/28

(52) U.S. Cl. CPC A61K 31/165; A61K 31/122; A61K 31/155; A61K 31/198; A61K 31/205; A61K 31/355; A61K 31/4045; A61K 31/593; A61K 33/06; A61P 25/28

(57) ABSTRACT

The present disclosure relates to a therapeutic composition for treating, preventing, inhibiting, improving, or slowing down a neurodegenerative disease, in particular a disease related to aggregate formation of TAR DNA-binding protein 43(TDP-43) such as amyotrophic lateral sclerosis, or the onset or development of such condition, by using a combination agent containing colchicine or a pharmaceutically acceptable salt thereof and metformin or a pharmaceutically acceptable salt thereof as active ingredients. The inventors of the present disclosure have confirmed that neurodegenerative diseases as described above may be treated or prevented by using a combination agent in which colchicine and metformin, which are drugs not used in the related art, are mixed as a therapeutic agent for conditions as described above.

### CROSS REFERENCE TO RELATED APPLICATIONS

This application is a National Stage of International Application No. PCT/KR2023/016948, filed on Oct. 29, 2023, which claims priority from Korean Patent Application No. 10-2022-0144058, filed on Nov. 1, 2022.

### TECHNICAL FIELD

The present disclosure relates to a therapeutic composition for treating, preventing, inhibiting, improving, or slowing down a disease related to aggregate formation of TAR DNA-binding protein 43 (TDP-43), or the onset or development of such condition, by using a combination agent containing colchicine or a pharmaceutically acceptable salt thereof and metformin or a pharmaceutically acceptable salt thereof as active ingredients.

The conditions related to the TDP-43 protein and the like, as described above, are mainly neurodegenerative diseases, including Frontotemporal dementia (FTD), Amyotrophic lateral sclerosis (ALS), Alzheimer's disease (AD), Parkinson's disease (PD), Chronic traumatic encephalopathy (CTE), Limbic-predominant age-related TDP-43 encephalopathy (LATE) and the like, and include diseases or disorders and abnormalities related to TDP-43, particularly related to TDP-43 aggregates, or TDP-43 proteinopathy and the like.

The inventors of the present disclosure have confirmed that neurodegenerative diseases as described above may be treated or prevented by using a combination agent in which colchicine and metformin, which are drugs not used in the related art, are mixed as a therapeutic agent for conditions as described above.

### BACKGROUND ART

Neurodegenerative diseases typified by Parkinson's disease, Alzheimer's disease, stroke, and the like are known to be ultimately caused by cell death leading to neuronal damage and loss. For example, in the case of Parkinson's disease, dopaminergic neurons in the melanoma of the midbrain are selectively lost, resulting in dysfunction of the basal ganglia involved in motor function tissue, leading to motor dysfunction such as persistent tremor, stiffness, frostbite, postural instability, etc.

In such a neurodegenerative disease, the inventors of the present disclosure have confirmed that a combination agent combining colchicine and metformin is particularly effective in amyotrophic lateral sclerosis (ALS), also known as Lou Gehrig's disease, which is highly related to TDP-43 protein.

ALS, also called Lou Gehrig's disease, is a rare neurodegenerative disease in which progressive degeneration of motor neurons occurs in the central nervous system, characterized by death of neurons in motor neurons. Studies on the precise pathogenesis and therapeutics of ALS have not yet been clearly identified, and the drugs currently approved as therapeutics for ALS by the U.S. FDA are Riluzole and Edaravone component preparations, both of which are known to help extend life expectancy in patients by several months or delay the progression of the disease at an early stage, but there is still no fundamental treatment for ALS.

It has been reported that about 10% of patients with ALS have a genetic cause, and it is estimated that 90% of the remaining patients have a cause such as immunity, oxidative toxicity, excitotoxicity, environmental factors, or protein aggregation.

Among them, aggregation of a protein called TDP-43 has been observed in about 90% or more of ALS patients, and many studies have reported that this phenomenon is an important cause of the development of Lou Gehrig.

TDP-43 is mainly present in the cell nucleus, and it is known that the accumulation of TDP-43 aggregates in the cytoplasm causes TDP-43 proteinopathy, and abnormal aggregation of TDP-43 in the cytoplasm has recently been found in the brain of patients with limbic-predominant age-related TDP-43 encephalopathy (LATE), Alzheimer's disease (AD).

Developing effective treatments for ALS as the main therapeutic target in the present disclosure is causing considerable difficulties in the pharmaceutical industry as a clear mechanism for causing ALS disease has not yet been identified as described above.

Riluzole (RILUTEK®, Sanofi) and Edaravone (RADICUT®, Mitsubishi Tanabe Pharma) are currently the only approved therapeutic agents for ALS, but Riluzole is a neuroprotective drug that blocks glutamic acid neurotransmission in the central nervous system to prevent Apoptosis (programmed cell death) of motor neurons, and Edaravone inhibits oxidative stress caused by free radicals, and all of these therapeutic agents have been confirmed to have only a symptom-relieving effect.

Therefore, there is a need to develop improved therapies for the treatment of TDP-43 proteinopathy, such as ALS, and developing such effective therapies will not only contribute to improving the quality of life and longevity of patients suffering from ALS disease, but will also significantly help to lower the cost burden of such diseases.

With respect to TDP-43 proteinopathy, as provided in the present disclosure, no studies have yet been conducted on a therapeutic method using a combination agent containing colchicine and metformin.

### PRIOR ART DOCUMENTS

### Patent Document

Korean Patent Publication No. 10-2012-0023352

Korean Patent Publication No. 10-2022-0120906

### CONTENT OF THE INVENTION

### Technical Problem

As described above, the present disclosure is intended to solve a situation in which a therapeutic agent for treating ALS related to TDP-43 proteinopathy is insufficient, and clear research on the disease is not sufficiently conducted, so that it is a considerable burden to develop a new therapeutic agent.

Accordingly, in the present disclosure, as a result of continuous research on the therapeutic effect on the disease using a combination agent in which colchicine and metformin are mixed, which has not been studied as a therapeutic agent for ALS in the related art, it has been confirmed that the combination agent has a sufficient effect, thereby completing the present disclosure.

Age-related brain disorders characterized by pathological aggregation of proteins in the central nervous system (CNS) and peripheral organs are one of the leading causes of disability and death worldwide. The protein that forms the best characterized aggregates in Alzheimer's disease and related disorders is Amyloid-β. Other disease-related, aggregation-prone proteins that cause neurodegeneration are, but are not limited to, α-synuclein (αSyn), Huntingtin, Fused in sarcoma (FUS), Dipeptide repeat protein (DPR) generated by unconventional translation of C9orf72 repeat expansions, Superoxide dismutase 1 (SOD1), and TDP-43. Among these, diseases related to TDP-43 aggregates are generally referred to as TDP-43 proteinopathies, including, without limitation, ALS and FTD.

Transactive response DNA-binding protein 43 kDa (TDP-43) is a 414-amino acid protein encoded by the TARDBP gene on chromosome 1p36.2 (ALS10). TARDBP consists of six exons (exon 1 is non-coding; exons 2-6 are protein-coding). TDP-43 belongs to the family of heteronuclear ribonucleoprotein (hnRNP) RNA binding proteins. TDP-43 includes five functional domains. It consists of two RNA recognition motifs (RRM1 and RRM2) with two highly conserved hexameric ribonucleoprotein 2 (RNP2) and octameric ribonucleotide 1 (RNP1) regions, a nuclear export signal (NES) and a nuclear localization signal (NLS) that allow reciprocation between the nucleus and cytoplasm transporting the bound mRNA and a C-terminal glycine rich domain that mediates protein-protein interactions, and the like.

TDP-43 has been implicated in several aspects of RNA processing, including transcription, splicing, transport, and stabilization. It is a highly conserved ubiquitously expressed protein with strictly self-regulated expression levels that reciprocate continuously between the nucleus and cytoplasm, but is predominantly localized within the nucleus. In 2006, TDP-43 was identified as a protein that accumulates in most FTLD and ALS outbreaks with tau-negative, ubiquitin-positive inclusion bodies (referred to as FTLD-TDP).

Thirty-eight negative-dominant mutations of TDP-43 were identified in sporadic and familial ALS patients as well as in hereditary FTD patients located primarily in the glycine rich domain. TDP-43 is inherently prone to aggregation as shown by sedimentation analysis, and this trend is further augmented by a portion of ALS-related TARDBP mutations that link TDP-43 aggregation to clinical disease manifestations.

In a TDP-43 proteinopathy cell model, combination agent of colchicine and metformin increased the activity of autophagy and chaperone, reducing abnormal aggregation of TDP-43 protein or accumulation of TDP-43 protein in the cytoplasm by mislocalization, and no such results have been reported so far.

Autophagy is known to play an important role in maintaining homeostasis of cells that decompose unnecessary proteins or abnormal organelles in cells and recycle them as new energy sources, and it is also known that such a deficiency or functional abnormality of autophagy is closely related to various human diseases including brain diseases.

On the other hand, chaperone, which is involved in preventing protein misfolding and suppressing damage and death of neurons, is well known as a defense mechanism against protein aggregation and has been regarded as an important target in the field of treating neurodegenerative diseases.

The inventors of the present disclosure first found that a composite composition of colchicine and metformin may effectively inhibit abnormal aggregation of the TDP-43 protein, which is a major etiological factor of neurodegenerative diseases, and further confirmed that the composition of the present disclosure exhibits a mechanism of selectively inducing chaperone and autophagy related to neurodegenerative disorders.

It is an object of the present disclosure from these results to provide a combination composition of colchicine and metformin for treating or preventing ALS related to TDP-43 protein. In addition to ALS, the present disclosure aims to provide a pharmaceutical composition for preventing or treating disease related to aggregation of TDP-43 protein, neurodegenerative brain diseases such as Frontotemporal dementia (FTD), Alexander disease, argyrophilic grain disease (AGD), ALS/Parkinsonism-dementia complex of Guam, Limbic-predominant age-related TDP-43 encephalopathy (LATE), Chronic traumatic encephalopathy (CTE), Parry-Romberg syndrome, Alzheimer's disease (AD), Down syndrome, Familial British dementia (FBD), Huntington's disease (HD), Machado-Joseph disease (MJD), Hippocampal sclerosis dementia (HSD), Inclusion-body myositis, Oculopharyngeal muscular dystrophy (OPMD), Myofibrillar myopathy (MFM), Traumatic brain injury, Dementia with Lewy bodies (DLB) and Parkinson's disease (PD).

The technical problem to be solved by the present disclosure is not limited to the above-mentioned problem, and various problems associated with the above-mentioned problems may be clearly understood by those skilled in the art from the following description.

### Technical Solution

In order to achieve the object, the present disclosure provides a pharmaceutical composition for the prevention or treatment of neurodegenerative brain diseases related to TDP-43 protein, including colchicine or a pharmaceutically acceptable salt thereof and metformin or a pharmaceutically acceptable salt thereof as main active ingredients, including Amyotrophic lateral sclerosis (ALS), Parkinson's disease, Alzheimer's disease or Pick's disease, Creutzfeldt-Jakob disease, Huntington's disease, progressive supranuclear palsy, spinocerebellar degeneration, cebellar atrophy, multiple sclerosis, senile dementia caused by loss of neurons, stroke, post-traumatic stress disorder, amnesia.

The present disclosure also provides a pharmaceutical composition for enhancing the therapeutic effect of the diseases, including colchicine or a pharmaceutically acceptable salt thereof and metformin or a pharmaceutically acceptable salt thereof as main active ingredients. The pharmaceutical composition for enhancing the therapeutic effect is the same concept as the therapeutic aid for preventing and treating ALS and the like, which are the target diseases of the present disclosure.

The pharmaceutical composition of the present disclosure may be prepared in the form of a pharmaceutical composition for the treatment or prevention of neurodegenerative diseases, further including an appropriate carrier, excipient or diluent commonly used in the preparation of pharmaceutical compositions, wherein the carrier may include a non-naturally occurring carrier. Specifically, the pharmaceutical compositions may be formulated and used in the form of powders, pills, granules, tablets, capsules, oral liquids, emulsified agent, suspensions, emulsions, syrups, aerosols, external preparations, sterile aqueous solutions, non-aqueous solvents, lyophilized preparation, suppositories, and sterile injections, respectively, according to conventional methods.

The pharmaceutical composition provided by the present disclosure may further include one or more components selected from the group consisting of melatonin, creatine, magnesium, coenzyme Q10, vitamin D, vitamin B, vitamin E, L-serine, L-carnitine and N-acetylcystein, which are effective in neurodegenerative diseases, in addition to an appropriate pharmacologically acceptable carrier.

The pharmaceutical composition of the present disclosure may be administered in a pharmaceutically effective amount, the term “pharmaceutically effective amount” of the present disclosure means an amount sufficient to treat or prevent a disease at a reasonable benefit/risk ratio applicable to medical treatment or prevention, and the effective dose level may be determined according to the severity of the disease, the activity of the drug, the age, weight, health, sex of the patient, the sensitivity of the patient to the drug, the time of administration, the route of administration and the rate of excretion of the composition of the present disclosure used, the duration of the treatment, the factors including the drug to be combined or used concomitantly with the compositions of the present disclosure used and other factors well known in the medical arts. The pharmaceutical compositions of the present disclosure may be administered as separate therapeutic agents or in combination with other therapeutic agents and may be administered sequentially or simultaneously with conventional therapeutic agents, and may be administered singly or multiple times. In view of all of the factors, it is important to administer an amount that may achieve the maximum effect in a minimum amount without side effects.

The dosage of the pharmaceutical composition provided by the present disclosure may be determined by a person skilled in the art in consideration of the purpose of use, the degree of addiction to a disease, the age, weight, sex, previous history of patient, or the type of substance used as an active ingredient, and the like, but it is important to administer an amount that may achieve the maximum effect in a minimum amount without side effects in consideration of all of the factors.

### Effect of Invention

The pharmaceutical composition provided by the present disclosure containing colchicine or a pharmaceutically acceptable salt thereof and metformin or a pharmaceutically acceptable salt thereof as active ingredients inhibits abnormal aggregation and mislocalization of the TDP-43 protein due to the synergistic effect of the two components, and may efficiently prevent or treat neurodegenerative diseases such as ALS.

The combination preparation provided by the present disclosure may efficiently enhance the effect of preventing or treating the disease even when administered at a low dose without using a conventional expensive drug, and will greatly contribute to the promotion of human health and the development of related industries such as healthcare.

### BRIEF DESCRIPTION OF THE DRAWINGS

FIG. 1 quantifies mRNA expression (A-C) or protein expression (D) of chaperone HSPB8 in human SH-SY5Y cells when reagent-treated with either metformin or colchicine alone or with metformin/colchicine in combination.

FIG. 2 quantifies the aggregation of TDP-43 using a TDP-43 aggregation kit in H₂O₂-treated human SH-SY5Y cells when reagent-treated with either metformin or colchicine alone or with metformin/colchicine in combination.

FIG. 3 shows (A) the results observing the degree of TDP-43 aggregation in H₂O₂-treated human SH-SY5Y cells when reagent-treated with either metformin or colchicine alone or with metformin/colchicine in combination, through immunofluorescence staining and fluorescence microscopy; (B) quantification of the stress granules measured. In addition, it shows (C) the confirmation and quantification of the degree of TDP-43 aggregation using a filter retardation assay.

FIG. 4 shows (A) fluorescence microscopy images showing the intracellular localization of TDP-25 in SH-SY5Y cells transfected with human GFP-TDP-25 when reagent-treated with either metformin or colchicine alone or with metformin/colchicine in combination; (B) the observed expression levels of insoluble TDP-43 and soluble TDP-43 proteins; and (C) the quantification of degree of TDP-43 aggregation. In FIG. 4, blue fluorescence indicates the nucleus with DAPI, and green fluorescence indicates TDP-25 with GFP.

FIG. 5 quantifies autophagy transcription factor TFEB mRNA expression in human SH-SY5Y cells when reagent-treated with either metformin or colchicine alone or with metformin/colchicine in combination.

FIG. 6 quantifies autophagy receptor SQSTM1/p62 mRNA expression in human SH-SY5Y cells when reagent-treated with either metformin or colchicine alone or with metformin/colchicine in combination.

FIG. 7 quantifies autophagy marker MAP1LC3A mRNA expression in human SH-SY5Y cells when reagent-treated with either metformin or colchicine alone or with metformin/colchicine in combination.

FIG. 8 quantifies p62 protein expression in human SH-SY5Y cells when reagent-treated with either metformin or colchicine alone or with metformin/colchicine in combination.

FIG. 9 quantifies LC3-II protein expression in human SH-SY5Y cells when reagent-treated with either metformin or colchicine alone or with metformin/colchicine in combination.

### DETAILED DESCRIPTION FOR CARRYING OUT THE INVENTION

It has been confirmed that the degree of aggregation of TDP-43 decreases when a combination agent provided by the present disclosure, which is formed by containing colchicine or a pharmaceutically acceptable salt thereof and metformin or a pharmaceutically acceptable salt thereof as an active ingredient, is administered to human SH-SY5Y cells, and the present disclosure has been completed based on this.

Hereinafter, the present disclosure will be described in detail.

Colchicine, which is a major active ingredient used in the present disclosure, is a substance extracted from plants of the genus Colchicum, and is a drug mainly used as an alternative drug for gout when a non-steroidal anti-inflammatory agent does not show a smooth therapeutic effect on gout, and is also used in the treatment of familial mediterranean fever, pericarditis, Behcet's disease, and the like.

Metformin, another active ingredient used in the present disclosure, is a biguanide drug that has an effect of improving blood glucose and is mainly used as a therapeutic agent for diabetes. It is known that the mechanism of action of metformin, although not exact, inhibits cellular respiration occurring in mitochondria and activates AMPK to prevent an increase in the concentration of cAMP induced by glucagon and consequently inhibits the activity of PKA to inhibit the production of glucose.

The diseases related to expression of the TDP-43 protein described in the present disclosure mean frontotemporal dementia [FTD such as those with or without sporadic or familial motor-neuron disorders, those with progranulin gene (GRN) mutations, those with C9orf72 mutations, those with TARDBP mutations, those with mutations in valosin-containing protein (VCP), those linked to chromosome 9p, corticobasal degeneration, frontotemporal lobar degeneration (FTLD) with ubiquitin-positive TDP-43 inclusion bodies (FTLDTDP), argyrophilic grain disease, Pick's disease, semantic variant primary progressive aphasia (svPPA), behavioral variant FTD (bvFTD), nonfluent variant primary progressive aphasia (nfvPPA), etc.], amyotrophic lateral sclerosis [ALS such as sporadic ALS, those with TARDBP mutations, those having angiogenin mutations], Alexander disease, Limbic-predominant age-related TDP-43 encephalopathy, Chronic traumatic encephalopathy, Parry-Romberg syndrome, Alzheimer's disease (including sporadic and familial forms of AD), Down syndrome, Familial British dementia, Polyglutamine disease [Huntington's disease and spinocerebellar ataxia type 3 (SCA3; also known as Machado-Joseph disease)], Hippocampal sclerosis dementia and myopathy (sporadic inclusion body myositis, inclusion-body myositis with mutations in valosin-containing protein); also Paget's disease of bone and frontotemporal dementia), Oculopharyngeal muscular dystrophy, Myofibrillar myopathy with mutation in Myotilin gene (MYOT) or mutation in gene encoding Desmin (DES), Traumatic brain injury, Dementia with Lewy bodies, Parkinson's disease, or the like.

The inventors of the present disclosure have confirmed through various experiments that abnormal aggregation of the TDP-43 protein is reduced when colchicine and metformin are reagent-treated in combination form.

In one embodiment of the present disclosure, the amount of inhibition of TDP-43 protein aggregation was compared in SH-SY5Y cells, which are human neuroblastoma cell lines, administered with colchicine alone, metformin alone, or colchicine and metformin in combination, the greatest reduction in aggregation of the protein was observed in administration with colchicine and metformin in combination.

In addition, in one embodiment of the present disclosure, when colchicine and metformin are administered in combination to the cell line, abnormal aggregation of TDP-43 induced by oxidative stress is much more inhibited than when the drug is administered alone.

In addition, in one embodiment of the present disclosure, when colchicine and metformin were administered in combination in the TDP-43 aggregation-cell model, the aggregation of TDP-43 was significantly inhibited compared to when the drug was administered alone, and the results showed that administration with colchicine and metformin in combination inhibits abnormal aggregation of TDP-43 by increasing the expression of autophagy marker genes and chaperone proteins in the cells.

From the results, it was confirmed that the pharmaceutical composition provided by the present disclosure increases the expression of chaperone gene, increases the expression of autophagy gene, and inhibits the abnormal aggregation and mislocalization of TDP-43 protein in the disease mediated by abnormal aggregation of TDP-43 protein or accumulation of TDP-43 protein in the cytoplasm by mislocalization, and through this, the present inventors completed the present disclosure.

The term “combination agent” as used herein means the pharmaceutical composition provided by the present disclosure.

The compositions of the present disclosure may further include suitable carriers, excipients and diluents commonly used in the preparation of pharmaceutical compositions. In addition, it may be formulated and used in the form of powders, pills, granules, tablets, capsules, oral liquids, emulsified agent, suspensions, emulsions, syrups, aerosols, external preparations, sterile aqueous solutions, non-aqueous solvents, lyophilized preparation, suppositories, and sterile injections, according to conventional methods, and may be preferably formulated and used as a unit dosage form preparation suitable for oral administration.

The pharmaceutical composition according to the present disclosure is administered in a pharmaceutically effective amount. In the context of the present disclosure, a “pharmaceutically effective amount” means an amount sufficient to treat a disease at a reasonable benefit/risk ratio applicable to medical treatment, and the effective dose level may be determined according to the type, severity, activity of the drug, sensitivity to the drug, time of administration, route of administration and rate of excretion, duration of treatment, factors including concurrently used drugs, and other factors well known in the medical arts of the patient. The pharmaceutical compositions according to the present disclosure may be administered as individual therapeutic agents or in combination with other therapeutic agents and may be administered sequentially or simultaneously with conventional therapeutic agents, and may be administered singly or multiply. In view of all of the factors, it is important to administer an amount that may achieve the maximum effect in a minimum amount without side effects, which may be easily determined by a person skilled in the art.

The pharmaceutical compositions of the present disclosure may be administered to a subject by a variety of routes. All modes of administration may be envisaged, e.g., by oral, rectal or intravenous, intramuscular, intraperitoneal, subcutaneous, intranasal, transdermal, intrauterine dural or intra-cerebrovascular injection. The pharmaceutical composition of the present disclosure is determined according to the type of drug that is the active ingredient, together with various related factors such as the disease to be treated, the route of administration, the age, sex and weight of the patient and the severity of the disease.

Preferred embodiments are shown below to facilitate understanding of the present disclosure. However, the following examples are only provided for easier understanding of the present disclosure, and the contents of the present disclosure are not limited by the following examples.

### EXAMPLE 1

### Whether the Combination Treatment of Metformin and Colchicine Exhibits a Synergistic Effect on the Increase in Expression of the Chaperone Protein HSPB8

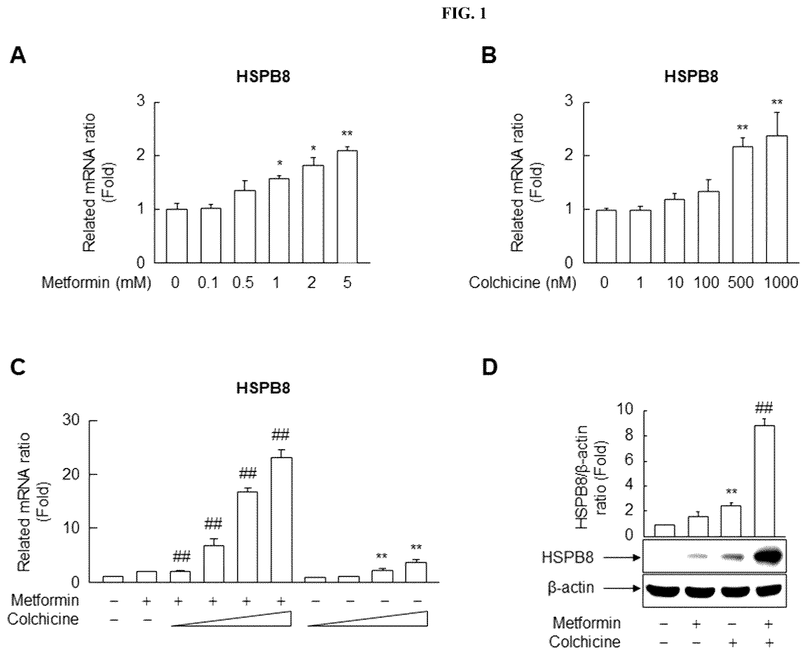

The SH-SY5Y cells, which are human neuroblastoma cell lines, were reagent-treated with metformin (0, 0.1, 0.5, 1, 2, 5 mM) or colchicine (0, 1, 10, 100, 500, 1000 nM) at each concentration, and 24 hours later, mRNA expression of the chaperone protein HSPB8 (Heat Shock Protein Family B (Small) Member 8) was observed, and the results are shown in FIG. 1.

It could be observed that the mRNA expression of HSPB8 was increased by 20-fold or more in a concentration-dependent manner with respect to colchicine when the reagent treatment of metformin (5 mM) and colchicine (10, 100, 500, 1000 nM) in combination was performed, compared to the approximately 2-fold increase in the mRNA expression induced by the reagent treatment with either metformin or colchicine alone (A to C of FIG. 1).

Since it was confirmed that the combination treatment of metformin and colchicine had an additive effect on the mRNA expression of HSPB8, the protein expression of HSPB8 was observed under fixed concentrations of metformin (5 mM) and colchicine (1000 nM). At this time, the protein expression of HSPB8 showed an inadequate change by the reagent treatment with either metformin or colchicine alone, but it was confirmed that there was an additive effect of approximately 8-fold increase by the combination treatment of metformin and colchicine (D of FIG. 1, **P<0.01, *P<0.05 in each figure are compared to the normal cell group; ^{##}P<0.01 is compared to the colchicine-only group).

### EXAMPLE 2

### Whether the Combination Treatment of Metformin and Colchicine Inhibits Abnormal Aggregation of TDP-43 Induced by Oxidative Stress

The SH-SY5Y cells, which are human neuroblastoma cell lines, were reagent-treated with metformin (0, 0.1, 0.5, 1, 2, 5 mM) or colchicine (0, 1, 10, 100, 500, 1000 nM) at each concentration, and 24 hours later, with 1 mM hydrogen peroxide (H₂O₂) for 1 hour to induce oxidative stress.

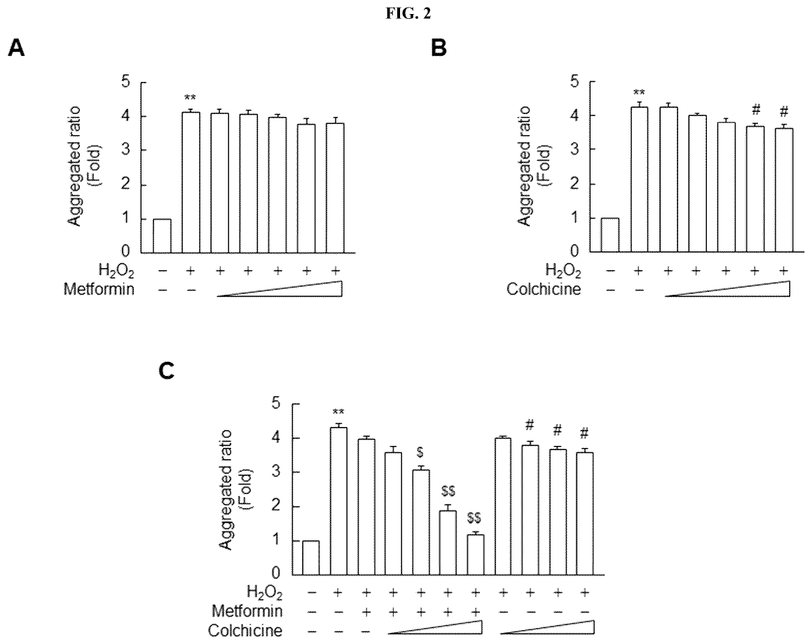

Subsequently, it was observed that degree of aggregation of TDP-43 in each sample was measured, and that 1 mM hydrogen peroxide (H₂O₂) significantly induced abnormal aggregation of TDP-43, and treatment with metformin alone did not have a significant effect on TDP-43 aggregation induced by hydrogen peroxide (A of FIG. 2), and it was confirmed that treatment with colchicine alone also showed a slight effect thereon (B of FIG. 2).

However, it was confirmed that hydrogen peroxide-induced TDP-43 aggregation was significantly inhibited in the reagent treatment with metformin and colchicine in combination as compared to the reagent treatment with the drug alone, and in particular, it was observed that the group of combination treatment with highest concentration of colchicine (1000 nM) and metformin was inhibited to almost normal cell level (C of FIG. 2).

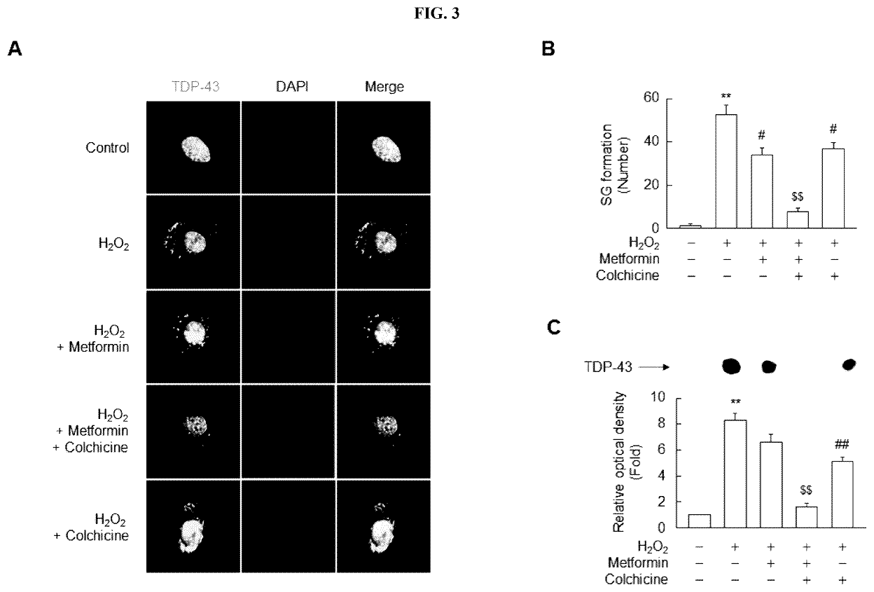

The results shown in FIG. 2 were confirmed once again using a microscope and cellular proteins (FIG. 3). It was confirmed that hydrogen peroxide (H₂O₂) significantly induced TDP-43 aggregation in the cytoplasm, and treatment with either metformin or colchicine alone did not inhibit TDP-43 flocculation in the cytoplasm, but reagent treatment with metformin and colchicine in combination inhibited the aggregation of TDP-43 to the level of normal cells (A and B of FIG. 3). This result could be confirmed once again in the filter retardation assay experiment of C of FIG. 3 (In the figures, ** P<0.01 is compared to the normal cell group; ##P<0.01, #P<0.05 are compared to the hydrogen peroxide group; ^{$$}P<0.01, ^{$}P<0.05 are compared to hydrogen peroxide+colchicine only group).

### EXAMPLE 3

### Whether the Combination Treatment of Metformin and Colchicine Inhibits Aggregation of TDP-43 in TDP-43 Aggregated-Cell Model

To construct an aggregation model of TDP-43 protein in the SH-SY5Y cells, which are human neuroblastoma cell lines, a GFP-TDP-25 plasmid was transfected into cells, and it may be confirmed that the transfected GFP-TPD-25 (GFP: green fluorescent protein) is present in the cytoplasm.

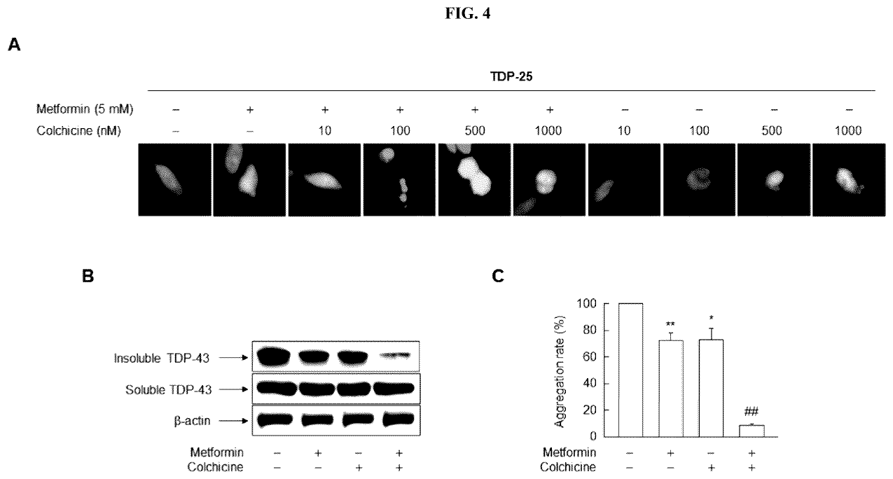

It was confirmed that treatment of the cells with either metformin or colchicine alone did not affect abnormally present TDP-25 in the cytoplasm, whereas reagent treatment with metformin and colchicine in combination inhibited accumulation of TDP-25 in the cytoplasm (A of FIG. 4).

In addition, it was confirmed that, in the TDP-43 aggregation model using the GFP-TDP-25 plasmid, treatment with either metformin (5 mM) or colchicine (1000 nM) alone slightly reduced the expression of insoluble TDP-43, whereas reagent treatment with metformin and colchicine in combination significantly reduced the expression of Insoluble TDP-43 (B and C of FIG. 4, in the figures, **P<0.01, *P<0.05 are compared to the normal cell group; and ^{##}P<0.01 is compared to the colchicine-only group).

### EXAMPLE 4

Whether the Combination Treatment of Metformin and Colchicine Exhibits a Synergistic Effect on the Increase in Autophagy of the Cell

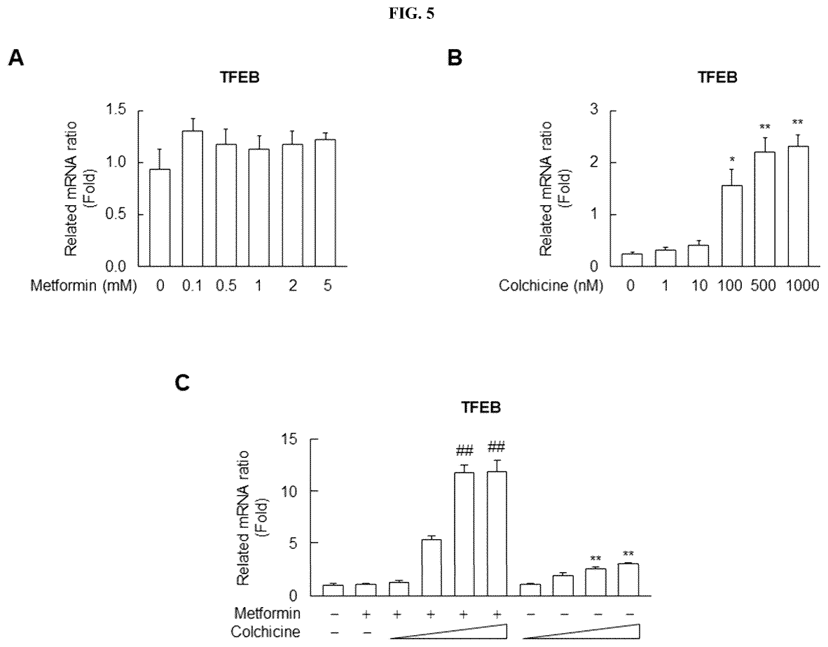

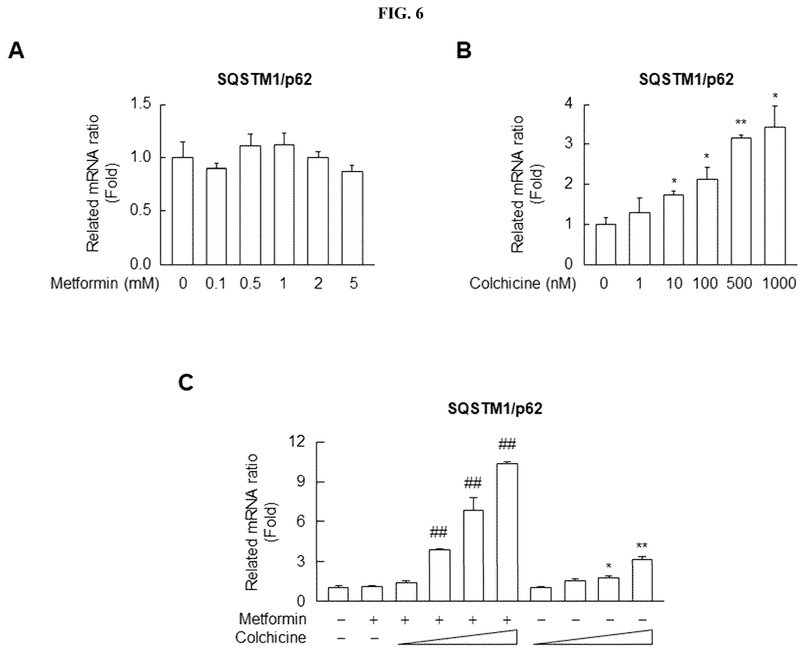

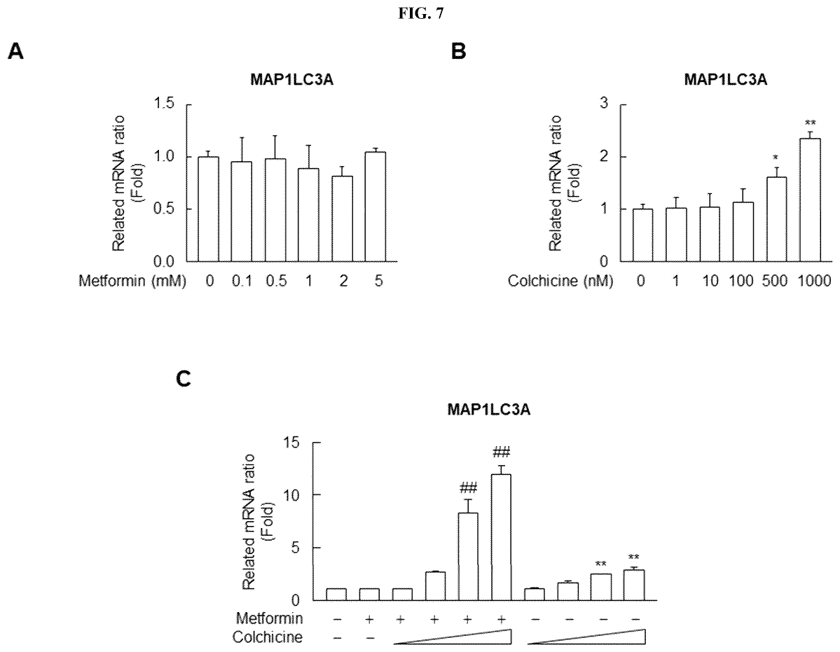

The SH-SY5Y cells, which are human neuroblastoma cell lines, were reagent-treated with metformin (0, 0.1, 0.5, 1, 2, 5 mM) or colchicine (0, 1, 10, 100, 500, 1000 nM) at each concentration, and 24 hours later, mRNA expressions of TFEB (FIG. 5), which is an autophagy master transcription factor, SQSTM1/p62 (FIG. 6), which is an autophagy receptor, and the autophagy marker MAP1LC3A (FIG. 7) were observed.

In the mRNA expression changes of the TFEB, SQSTM1/p62 and MAP1LC3A genes, it was confirmed that the reagent treatment with metformin alone showed no effect, and the reagent treatment with colchicine alone showed an increase effect of about 2 to 3 times. In comparison, it could be observed that the reagent treatment with metformin (5 mM) and colchicine (10, 100, 500, 1000 nM) in combination had a 10-fold or more additive effect on the expression of the genes (FIGS. 5, 6, and 7).

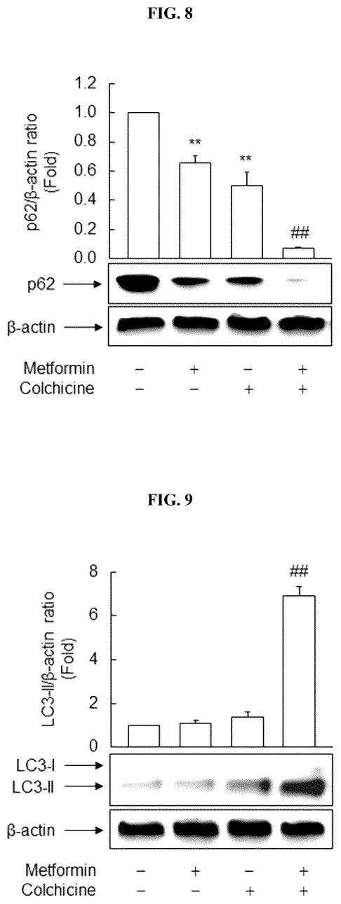

In addition, to confirm the effect of metformin and colchicine on autophagy in the TDP-43 aggregation model, SH-SY5Y cells transfected with the GFP-TDP-25 plasmid were reagent-treated with metformin (5 mM) and colchicine (1000 nM) alone or in combination. It was confirmed that the protein expression of p62 increased by the reagent treatment was significantly inhibited by the reagent treatment with metformin and colchicine in combination compared to the reagent treatment with either metformin or colchicine alone (FIG. 8). In addition, it was confirmed that LC3-II expression was significantly increased by the reagent treatment with metformin and colchicine in combination, compared to the reagent treatment with either metformin or colchicine alone (FIG. 9, wherein **P<0.01, *P<0.05 are compared to the normal cell group; ^{##}P<0.01, ^{#}P<0.05 are compared to the colchicine-only group).

As may be seen from the results, the abnormal aggregation of the TDP-43 protein was hardly reduced by reagent treatment with either metformin or colchicine alone, but the aggregation-inhibiting effect of the protein was significantly exhibited by the administration of colchicine and metformin in combination, which means that two drugs exhibited an obvious synergistic effect, enabling the efficient prevention or treatment amyotrophic lateral sclerosis such as Lou Gehrig's disease and the like.

The foregoing description of the present disclosure is intended to be illustrative, and it will be understood by those skilled in the art that present invention may be easily modified into other specific forms without altering the technical spirit or essential features of the present disclosure. It is, therefore, to be understood that the embodiments described above are illustrative in all respects and not restrictive.

### INDUSTRIAL APPLICABILITY

The present disclosure is industrially applicable.

1. A method for preventing or treating neurodegenerative disease, comprising administering to a subject in need thereof a pharmaceutical composition comprising colchicine or a pharmaceutically acceptable salt thereof, and metformin or a pharmaceutically acceptable salt thereof.

2. The method for preventing or treating neurodegenerative disease according to claim 1, wherein the neurodegenerative disease is a disease mediated by abnormal aggregation of TDP-43 protein or accumulation of TDP-43 protein in the cytoplasm by mislocalization.

3. The method for preventing or treating neurodegenerative disease according to claim 2, wherein the disease mediated by abnormal aggregation of TDP-43 protein or accumulation of TDP-43 protein in the cytoplasm by mislocalization is any one selected from the group consisting of Frontotemporal dementia (FTD), Amyotrophic lateral sclerosis (ALS), Alexander disease, Argyrophilic grain disease (AGD), ALS/Parkinsonism-dementia complex of Guam, Limbic-predominant age-related TDP-43 encephalopathy (LATE), Chronic traumatic encephalopathy (CTE), Parry-Romberg syndrome, Alzheimer's disease (AD), Down syndrome, Familial British dementia (FBD), Huntington's disease (HD), Machado-Joseph disease (MJD), Hippocampal sclerosis dementia (HSD), Inclusion-body myositis, Oculopharyngeal muscular dystrophy (OPMD), Myofibrillar myopathy (MFM), Traumatic brain injury, Dementia with Lewy bodies (DLB) and Parkinson's disease (PD).

4. The method for preventing or treating neurodegenerative disease according to claim 1, wherein the pharmaceutical composition increases chaperone gene expression, increases autophagy gene expression, and inhibits abnormal aggregation and mislocalization of TDP-43 protein, in the disease mediated by abnormal aggregation of TDP-43 protein or accumulation of TDP-43 protein in the cytoplasm by mislocalization.

5. The method for preventing or treating neurodegenerative disease according to claim 1, wherein the pharmaceutical composition is formulated for administration by a variety of routes including oral, rectal, intravenous, intramuscular, intraperitoneal, subcutaneous, intranasal, intrauterine, intra-cerebrovascular, and transdermal administration.

6. The method for preventing or treating neurodegenerative disease according to claim 1, wherein the pharmaceutical composition is prepared in any one of formulations selected from the group consisting of powders, pills, granules, tablets, capsules, oral liquids, emulsified agent, suspensions, emulsions, syrups, aerosols, external preparations, sterile aqueous solutions, non-aqueous solvents, lyophilized preparation, suppositories, and sterile injections.

7. The method for preventing or treating neurodegenerative disease according to claim 1, wherein the pharmaceutical composition further comprises one or more components selected from the group consisting of melatonin, creatine, magnesium, coenzyme Q10, vitamin D, vitamin B, vitamin E, L-serine, L-carnitine and N-acetylcystein.
